(19) United States
(12) Patent Application Publication
(10) Pub. No.: US 2017/0007591 A1
(43) Pub. Date: Jan. 12, 2017

(54) Composition to Mitigate Spikes in Blood Sugar

(71) Applicant: Panacea Scientific, Inc., West Hartford, CT (US)

(72) Inventors: Chad LaChapelle, East Longmeadow, MA (US); Tim Maloney, Avon, CT (US); Matt Smith, Westport, CT (US); Jerry Stanton, Farmington, CT (US)

(21) Appl. No.: 14/793,707

(22) Filed: Jul. 7, 2015

(51) Int. Cl. A61K 31/4525; A61K 36/9066; A61K 31/12; A61K 31/353; A61K 31/235

(52) U.S. Cl. CPC A61K 31/4525; A61K 31/353; A61K 31/235; A61K 31/12; A61K 36/9066

(57) ABSTRACT

Compositions comprising therapeutically effective amounts of quercetin, myricetin and chlorogenic acid are improved by the addition of one or multiple additional components, for mitigating blood sugar spikes in general and for treating diabetes specifically. Certain other additives produce additional benefits.

### FIELD OF THE INVENTION

Embodiments of these teachings relate to treatments for diabetes and other metabolic disorders, and more generally relate to mitigating spikes in blood sugar.

### BACKGROUND

Therapeutically effective amounts of quercetin, myricetin and chlorogenic acid in combination are known to be particularly useful in the treatment of obesity and diabetes, and to achieve weight loss and/or weight control. See for example US Patent Application Publication Nos. 2008/0234364, 2011/0118345, 2012/0252887, and 2014/0031420, of which the contents of each are incorporated herein by reference. Additionally, US Patent Application Publication No. 2009/0298932, also incorporated by reference, attests the benefits in treating neurological disorders such as Alzheimer's disease using similar combinations of these three components. Each of these components is naturally occurring, though they cannot be found in nature as the tri-part combination described.

The above references describe the following as therapeutically effective amounts:
a) about 0.5 to about 15% by weight quercetin; about 25% to about 65% by weight chlorogenic acid; and about 25% to about 65% by weight of myricetin, based on the total weight of the mixture;
b) about 0.5 to about 10% by weight quercetin; about 30% to about 60% by weight chlorogenic acid; and about 30% to about 60% by weight of myricetin, based on the total weight of the mixture;
c) about 0.5 to about 5% by weight quercetin; about 35% to about 55% by weight chlorogenic acid; and about 35% to about 55% by weight of myricetin, based on the total weight of the mixture;
d) a ratio of chlorogenic acid to quercetin to myricetin of about 1:(2-4):(2-4), or about 1:(2-3):(2-3), or about 1:3:3;
e) a ratio of quercetin to chlorogenic acid to myricetin of about 1:(20-75):(20-75), or about 1:(30-60):(30-60), or about 1:(40-55):(40-55);
f) for a dietary supplement: 5 to about 100 mg quercetin; about 100 to about 500 mg chlorogenic acid; and about 100 to about 500 mg myricetin; and
g) for a dietary supplement: about 5 to about 50 mg quercetin; about 250to about 400 mg chlorogenic acid; and about 250 to about 400 mg myricetin.

Increasingly larger percentages of adults and children are becoming overweight and developing diabetes or pre-diabetes. This problem is particularly acute in more advanced economies in which foods with high sugar and caloric content is plentiful and relatively inexpensive, and where adult work functions are generally less physically demanding than a t times previous to the present computer age. The above compounds of quercetin, myricetin and chlorogenic acid may be somewhat effective for improving the ratio between lean and fat tissue in humans, but the embodiments herein improve upon those results.

### SUMMARY

In one aspect of these teachings there is a composition comprising therapeutically effective amounts of quercetin, myricetin and chlorogenic acid in combination with curcumin. In a more particular embodiment the curcumin is in an amount no greater than 12 g. In a further embodiment the amount of curcumin is greater than 50 mg. In a still further embodiment the curcumin is in the form of an extract of turmeric root. Certain of these curcumin embodiments have the composition further comprising an agent that increases bioavailability of the curcumin in humans by at least 50%, for example piperine in an amount greater than 20 mg. In a particular embodiment the agent is in the form of black pepper. In another embodiment the composition is in combination with dried tea leaves, for example the composition and the dried tea leaves are combined in a flow through tea bag wherein content of the tea bag is characterized by no more than a trace amount of an agent that increases bioavailability of the curcumin in humans.

In another aspect of these teachings there is a composition comprising therapeutically effective amounts of quercetin, myricetin and chlorogenic acid in combination with capsicum. In a particular embodiment the capsicum is in an amount no greater than 8 g, but greater than 2 g. In one implementation the capsicum is in the form of an extract of a hot pepper. This composition can also further comprise green tea or an extract thereof, and in one example the green tea or the extract thereof is in an amount greater than 50 mg. In one example the above composition is in combination with dried tea leaves, for example combined in a flow through tea bag. The dried tea leaves may be dried green tea leaves.

### BRIEF DESCRIPTION OF THE DRAWINGS

FIG. 1 illustrates the molecular structure of Quercitin.

FIG. 2 illustrates the molecular structure of Myricetin.

FIG. 3 illustrates the molecular structure of Chlorogenic Acid.

### DETAILED DESCRIPTION

Metabolic syndrome can often lead to diabetes and is characterized by a group of metabolic risk factors in humans including elevated blood pressure; elevated triglyceride levels and depressed HDL cholesterol levels in the blood that lead towards arterial plaque accumulation (atherogenic dyslipidemia); glucose intolerance or insulin resistance evidenced by spikes and troughs in blood sugar levels throughout a given day; and excessive fat tissue in the abdomen area (central obesity).

While there is a genetic component to diabetes and related metabolic disorders in general, environmental risk factors that highly correlate to development of the disease include combinations of physical inactivity and diet that typically lead to elevated body weight due to excessive fat storage. Metabolic syndrome has become increasingly common in the United States, with some estimates at 20-25 percent of adults afflicted with this disorder that is closely correlated with insulin resistance whereby the body cannot efficiently utilize insulin.

Diabetes is used herein according to its general understanding in the medical arts and includes both type 1 and type 2 diabetes; generally high blood sugar (ketoacidosis), but also the more general chronic anomalies that arise from prolonged high blood sugar levels.

Therapeutically effective as used herein is a term of art in the pharmaceutical arts and includes curing an underlying disease or syndrome as well as mitigating its effects or extent to a measurable degree. The relevant biological effect may be local or systemic, and a therapeutically effective amount of a substance varies greatly depending upon the specific substance and the underlying disease/syndrome being addressed. Traditionally a therapeutically effective amount of a substance referred to an advantageous benefit/risk ratio across a sample population that can be extrapolated to the target population (e.g., adults with type 2 diabetes, all adults, etc.). In this traditional population-wide view for defining therapeutic effect, a therapeutically effective amount of a given substance for the sample population may be ineffective for a given individual due to genetic or environmental factors, and less than that same population-wide therapeutically effective amount may be highly effective in a different individual for similar reasons. Neither result undermines that the given amount is properly considered therapeutically effective so long as the advantageous benefit/risk ratio holds population-wide. In the current medical age of individualized medicine tailored for an individual person's specific genome, medical history, blood chemistry and sometimes even environmental factors, a therapeutically effective amount of a given substance can now be specific for a given individual.

It is well known that addressing the pathways of carbohydrate metabolism can mitigate diabetes and metabolic syndrome by means of weight loss in the subject. These carbohydrate metabolism pathways include inhibiting glucose absorption and glucose transport from the intestines to the bloodstream; enhancing glucose absorption and transport into muscle tissue; inhibiting gluconeogenesis; enhancing glucose liberation from fats; inhibiting carbohydrates being stored as fat; and inhibiting carbohydrate breakdown. Of these, the first four pathways are most relevant to the treatment of diabetes, though the first five are relevant for weight loss and weight control.

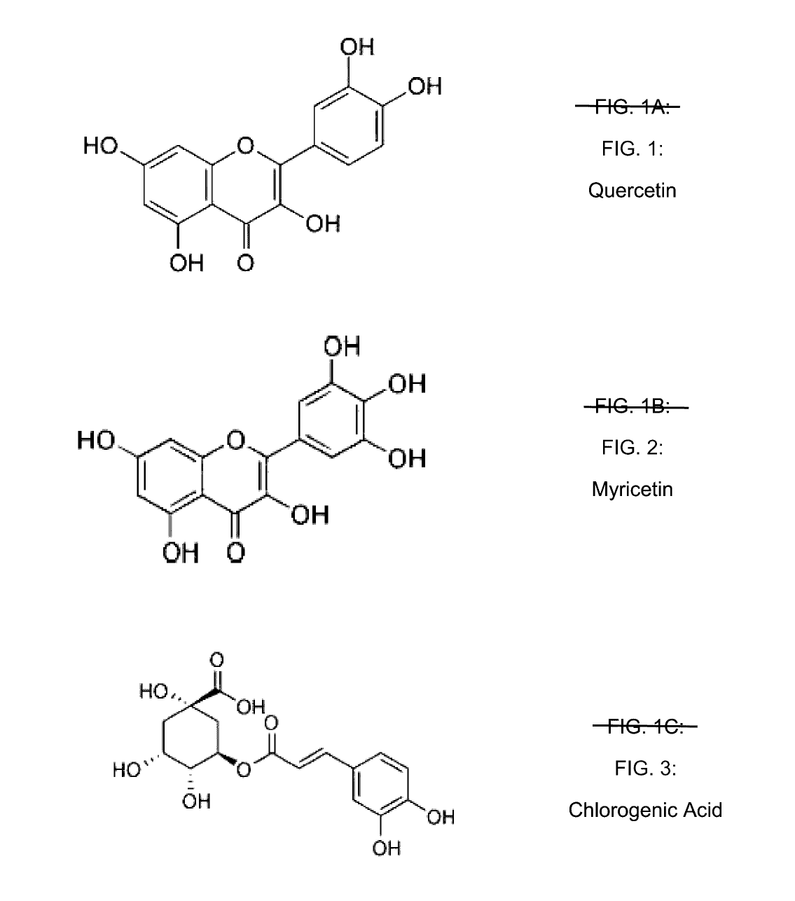

The references cited in the background describe that the combination of effective amounts of quercetin, myricetin and chlorogenic acid affects certain of the above metabolic pathways of carbohydrate metabolism, resulting in less glucose getting into the body and more glucose in the bloodstream getting shunted to the muscles. These are shown in the drawings: FIG. 1 is the Quercitin molecule, FIG. 2 is the Myricetin molecule, and FIG. 3 is the Chlorogenic Acid molecule. Certain of these references describe that when added to a carbohydrate-containing foodstuff in an effective amount, the composition of quercetin with myricetin and chlorogenic acid converts the foodstuff from a high glycemic index GI (i.e., 70 or more) food to a medium GI (i.e., 56-69) or low GI (i.e., 55 or less) food, making the foodstuff safer for diabetics to consume.

More specific to the metabolic pathways these component affect, these references describe that quercetin primarily inhibits GLUT2 transport inhibition which slows glucose absorption from the gut; secondarily causes glycogenolysis by lipid hydrolysis in that it releases glucose from adipose tissue; and thirdly inhibits fatty acid synthase (lipogenesis) which reduces the body's ability to store glucose as fat. As to myricetin, these references disclose that its relevant primary metabolic pathway is to inhibit glucosidase which inhibits the breakdown of starches resulting in less available carbohydrates; secondarily it stimulates the GLUT4 pathway which enhances the uptake of glucose into muscle and skeletal tissue resulting in less available glucose for storage as fat; and thirdly it inhibits the absorption of fructose.

And finally these references discuss that the relevant metabolic pathways of chlorogenic acid is primarily the inhibition of alpha-amylase which inhibits the breakdown of complex carbohydrates into transportable form thereby reducing the amount of carbohydrates that can be absorbed. Its secondary mechanism is the inhibition of Glucose 6 phosphate which reduces hepatic gluconeogenesis (e.g., the liver's ability to make glucose).

Embodiments of these teachings improve upon the above composition of quercetin, myricetin and chlorogenic acid by addition of further components. Additionally, these improved compositions can be delivered in a variety of comestible forms to better reach the wide variety of adults who suffer from diabetes and pre-diabetes, and to exploit additional benefits these improved compositions exhibit apart from the treatment of diabetes and metabolic disorders.

First consider embodiments directed primarily towards the treatment of diabetes. The prior art composition of quercetin, myricetin and chlorogenic acid, hereinafter referred to as QMCa for brevity, is improved in one embodiment by adding effective amounts of turmeric and/or capsaicin.

Turmeric technically refers to an extract of the turmeric plant, and is a common food spice for example by itself or as the main spice in curry. It is also used in some mustards, butters and cheeses, and turmeric root has an ancient pedigree with Indian ayurvedic medicine as well as traditional Chinese physicians. Turmeric has been used as for the treatment of liver and gallbladder problems, arthritis, headaches/fever, infections and inflammation, gas/bloating and other stomach pains, diarrhea, jaundice, and various other disorders. It is also reported for use as a topical skin treatment but these teachings presume turmeric is taken internally.

Curcumin is the yellow pigment associated with turmeric. It is a flavonoid polyphenol of the class curcuminoid. Curcumin is the ingredient of turmeric that exerts its anti-inflammatory effects, and this small molecule has been the focus of much research in the fields of cancer prevention and treatment.

Curcumin is also recognized for its positive effects on mitigating age-related cognitive decline, and for its dual effect in maintaining heart health by electrical means as reducing arterial lipid and plaque levels. This lipid and plaque removal, and to a greater extent curcumin's effect on reducing the risk of diabetes as well as side effects associated with that disease. Make it a suitable additive to the QMCa composition.

In general curcumin has a relatively poor bioavailability when taken orally; only a low percentage of the consumed volume is absorbed by the human body. This is due to its rapid metabolism in the liver and intestinal wall. But this bioavailability can be enhanced, for example with piperine which is the main alkaloid in black pepper and a known inhibitor of hepatic and intestinal glucuronidation. Piperine is known to inhibit several enzymes responsible for metabolizing nutritional substances, to stimulate amino-acid transporters in the intestinal lining, to inhibit removal of substances from cells so they continue to be available for use, and to decrease the intestinal activity allowing more of the substances to enter the body in active form. This latter mechanism is its primary advantage for pairing it with curcumin. Apart from black pepper piperine is also available as a supplement called bioperine.

Piperine dosages of 20 mg/kg in mice and humans has been shown to increase the serum concentration of a 2 g/kg dose of curcumin for up to two hours while greatly reducing the half life and clearance rate of curcumin. Specifically, the above 20 mg piperine dose has been shown to increase the bioavailability of curcumin by 154% in general for rats, and for humans it increased bioavailability of a 2 g dose of curcumin by 2000% during the first hour.

Since this increases absorption the addition of piperine generally reduces the anti-inflammatory effects of curcumin in humans since higher absorption yields less curcumin in the colon where it also helps with digestion. Curcumin has been shown to have a quite large safety threshold and only very limited and minor adverse side effects have been shown in humans at dosage levels up to 12 g curcuminoids per day. Piperine is also quite safe over a large dosage range, and 20 mg/kg should be considered as about the lower bound dosage if the purpose is to increase bioavailability of curcumin.

As a an additional component to QMCa these embodiments include turmeric extract 95% in the amount of 50-220 mg and preferably about 50-150 mg (or substantially equivalent dosages if the added extract is other than 95% pure turmeric, such as 52-158 or 52-232 mg pure turmeric extract). But as noted above dosages much higher than this are safe for human consumption, even up to 12 g curcumin per day. Preferably this is paired with at least 20 mg piperine to increase the uptake of the curcumin amounts. Progressively smaller amounts of piperine would still be beneficial but would yield a correspondingly reduced improvement over the above 2000% bioavailability increase for the curcumin.

Alternative to piperine, the bioavailability of orally administered curcumin has been shown to increase by 1200% when heated in boiling water for 10 minutes. This heating increases the otherwise-poor solubility of curcumin without evidence that this heating degrades its desirable properties, and it is the increased solubility which enables this type of dosage to overcome curcumin's otherwise rapid metabolism in the liver and intestinal wall. While impractical for some delivery vehicles, the composition of QMCa with curcumin/turmeric but without piperine is well suited for delivery via a hot beverage such as tea. In this case there is no need for piperine or other agents to increase the bioavalability of the curcumin; simply adding the QMCa composition with curcumin to dried tea leaves in a flow through tea bag will yield the beneficial result once the end user makes his/her tea.

Capsaicin is the principal pungent component found in habaneros, jalapenos, red chiles and other types of hot peppers. There are some conflicting research as to capsaicin's effect on cancer; some show it is a carcinogen while others show it acts as a cancer preventative. As an irritant capsaicin is biologically interesting in that it desensitizes the receptor cells; the neurons it initially excites become no longer responsive during a long lasting refractory period during which new neutrons are excited. This is a property that can be exploited for therapeutic purposes.

Like turmeric, capsaicin in the form of cayenne has an ancient history as a medicinal herb, specifically to relieve gastrointestinal disorders and other circulatory related syndromes. Cayenne has also been used as a first aid treatment for bleeding when other medical treatment is not immediately available, to relieve migraines and headaches, and to treat bladder dysfunctions. Its use as a topical treatment are not particularly relevant to these teachings and so will not be detailed, except to note that topical application has been shown to relieve the pain from diabetic neuropathy that originates from the nerves near the skin (similar to neuralgia). Diabetic neuropathy is a condition that occurs in some people with diabetes itself, but to the inventors' knowledge topical capsaicin has not been shown to cure diabetic neuropathy or diabetes.

Capsaicin is also an antioxidant that protects cells of the body from damage by harmful free radicals, is known for infection prevention, improving digestion, preventing heart disease, stimulating the cardiovascular system resulting in lower blood cholesterol levels and blood pressure, preventing atherosclerosis, and treating emphysema by thinning mucus to help move it out of the lungs and by strengthening lung tissue.

Capsaicin administered orally or by injection has also been shown effective in preventing obesity and facilitating weight loss, though the research on this score is not universally in agreement. Capsaicin in combination with green tea has been shown to suppress hunger and increased satiety, particularly during periods of negative energy balance when the body's caloric usage/burn is higher than its caloric intake. This hunger suppression and increased satiety may support weight loss which is a particularly important goal in the long term treatment of people with diabetes who have become overweight. When pairing with capsaicin or capsicum (see below), green tea should be in the range of 40-140 mg of 95%-purity green tea extract, though there are no adverse side effects known to the inventors for much larger dosage amounts.

Capsaicin is trans-8-methyl-N-vanillyl-5 nonenamide, a naturally occurring alkyl vanillylamide and a type of capsaicinoid (dihydrocapsaicin is another), typically produced by certain plants of the genus Capsicum (chile peppers) as a secondary metabolite. Species of capsicum include the chili pepper, the red pepper and paprika. Capsicum is the dry powder obtained by grinding up the fruits of these plants. Capsicum oleoresin (or capsaicin oleoresin) is the liquid concentrate extracted from that dry powder. Capsaicinoids are generally very soluble in fats, oils and alcohols. Pure capsaicin is a colorless and odorless crystalline to waxy compound, and it is capsicum that makes cayenne and other pepper extracts red.

Dosage amounts of 5 g capsicum have been shown to reduce blood glucose in young adults, and at high doses this reduction may be secondary to pancreatic stimulation that may induce insulin release. While both chili and turmeric are rich in phenols and would thus be expected to bind iron in the intestine and thus inhibit its absorption, in side by side experiments only chili was seen to do so despite that turmeric is far richer in phenolics. Iron absorption is an important health consideration particularly concerning women of child bearing age, and so the appropriate daily dose of capsaicin would appear to be upper bounded at about 8 g, and preferably no more than 5 g for women.

Another suitable additive to the above QMCa composition is apigenin in the amount of 1-35 mg/ml. Apigenin is a flavone of the class aglycone, specifically 4′,5,7-trihydroxyflavone, and has a yellow crystalline solid form. Traditionally it has been used as a dye for wool.

Apigenin has been reported to have chemopreventive properties; it induces a cellular waste recycling in leukemia cells (autophagy) though at the same time this may increase the subject's resistance to chemotherapy drugs such as vincristine. More generally it inhibits the enzyme CYP2C9 in humans, which is responsible for metabolizing vincristine and many other pharmaceutical drugs. Thus it may be appropriate when adding apigenin to the QMCa composition to place a warning label on the non-prescription product concerning the above drug effectiveness inhibitions. On the beneficial side apigenin is recognized as protecting against a wide variety of cancers, and it has a high selectivity for cancer cells as opposed to non-cancerous cells. It also has a very high safety threshold, and active doses for anti-cancer purposes can be obtained through consuming a vegetable and fruit rich diet, particularly parsley, celery and chamomile tea.

Apigenin is also known for preventing renal damage by reducing the expression of the cell death mediation bcl-2. One of the relatively rare properties of apigenin is that it activates monoamine transport. Like various other flavonoids, apigenin has nonomolar affinity for certain opioid receptors, including μ, δ-, and κ-opioid receptors and acts as a non-selective antagonist for each.

In a related vein apigenin as a supplement may be considered as a nutraceutical for its stimulation of adult neurogenesis that promotes neuronal differentiation. In this regard apigenin may be suitable for the treatment of certain neurological diseases, disorders and injuries, though at this time it appears the population-wide research to this effect is limited to animals.

Apigenin is found naturally in various plants including parsley, celery and chamomile tea leaves. The relatively high doses from consuming these plants has been shown to result in anxiety reduction in humans, and at still higher doses it may be considered as a mild sedative.

Another suitable additive to the above QMCa composition is chrysin in the amount of 200-600 mg. Chrysin is also a naturally occurring flavone, and is found naturally in certain flowers, chamomile tea leaves, certain mushrooms, and in honeycomb. Chrysin has been used as a bodybuilding supplement for its reported properties of raising testosterone production, but research to date has not proved this effect nor a link with raising estrogen levels in the body. While it generally has poor bioavailability (due to poor intestinal absorption followed by blockage at the cell membrane) and has been shown to produce detrimental effects (weight gain via increased fat storage) in the thyroid function of mice, it does possesses and anti-oxidation properties and suppresses liposacharide-induced COX-2 protein and mRNA expression which makes it potent as an anti-inflammatory. Chrysin has also been considered as a treatment for HIV/AIDS, erectile dysfunction and gout, but these treatments are not scientifically documented to the inventors' knowledge.

Clorius versicolor in the amount of 200-600 mg may also be a suitable additive to the above QMCa composition.

A further suitable additive to the above QMCa composition is 3,3′ diimdolylmethane or DIM, in the amount of 50-150 mg, or 2 mg/kg/day. DIM is produced in the human body from digesting certain cruciferous vegetables such as broccoli, brussels sprouts, cabbage and kale, and is likely the source of these vegetables being considered sop healthful. Specifically, DIM targets certain proteins to produce the following effects: anti-angiogenesis via HIF-1α; anti-viral/anti-cancer and anti-bacterial via IFN-γ and IFNGR; anti-inflammation via NF-κB, hormone control via ERβ; anti-androgen via AR; cytostatis via P38 and p21; and apoptosis via P13K and Akt. DIM is the subject of increased research lately due to its anti-cancer proclivities and to a lesser extent for its anti-radiation effects in preventing or reducing tissue damage via the NF-κB pathway due to radiation exposure.

Resveratrol 25% in the amount of 25% 50-150 mg (or equivalent dosages) is another suitable additive to the above QMCa composition. Resveratrol is a polyphenol with anti-oxidant properties and exhibits beneficial effects respecting cancer and heart disease. Resveratrol is naturally found in the skin of red grapes as well as peanuts and berries. It has become a popular supplement recently and many of these supplements in capsule form contain extracts of the Japanese and Chinese knotweed plants, as well as the more conventional sources of red grape extracts and red wine. Resveratrol has become quite popular to its anti-aging properties but it is also thought to possess weight reduction and anti-disease properties including protecting against heart disease by preventing oxidation of LDL cholesterol thus inhibiting arterial clotting, limiting the spread of cancer cells, protecting nerve cells from damage in Alzheimer's patients, and preventing insulin resistance in diabetics and pre-diabetics.

In this latter regard resveratrol is particularly suitable for adding to the above QMCa composition. It has been shown in mice to mimic the effects of caloric restriction and so may be suitable to counter the effects of unhealthy eating habits among humans, particular those with diabetes or pre-diabetes. Further research has shown the effect in humans is less pronounced than early studies would suggest, potentially due to a lack of NAD+ (nicotinamide adenine dinucleotide) and/or NADP+ to fully exploit the addition of reseveratrol to the system. This can be addressed by further adding niacinamide (also known as nicotinamide) and/or NMN (nicotinamide mononucleaotide) and/or NR (nicotinamide riboside), which are precursors to NAD and NADP production. Suitable amounts for each of these is up to 3 g/day.

Another suitable additive to the above QMCa composition is L-selinium methionine, or trace mineral selenium in the amount of 10-60 mg. Selenomethionine is a naturally occurring amino acid contianing selenium, and the L-enantiomer of selenomethionine is a common natural food source of selenium including in Brazil nuts, cereal grains, soybeans and legumes. Selenomethionine is an antioxidant due to its ability to deplete reactive species of oxygen. Selenomethionine is an organic form of selenium and thus is approximately 20% more bioavailable for the human body to absorb than the inorganic form selenite.

In one embodiment of the invention, the QMCa composition comprises about 5 to about 100 mg quercetin; about 100 to about 500 mg chlorogenic acid; and about 100 to about 500 mg myricetin. In another embodiment, the composition comprises about 5 to about 50 mg quercetin; about 250 to about 400 mg chlorogenic acid; and about 250 to about 400 mg myricetin.

The composition may be administered in the form of a dietary supplement, a food or beverage additive or as a pharmaceutical composition. In addition to quercetin, myricetin and chlorogenic acid, the composition may include one or more additives as discussed above by non-limiting examples.

The above additional components to the base quercitin-myricetin-chlorogenic acid compound can be individually or in any combination of two of more such additional components. There are various suitable pathways to administer such compositions of whatever makeup. In one embodiment described above for curcumin the composition was disposed in a tea bag with dried tea leaves, so that the heated water into which the end immerses this tea bag user acts to increase the bioavailabiliy of the curcumin. Any of the above compositions can be provided in this way, as well as in prepared iced tea though of course the iced version would not exploit the curcumin bioavailability increase.

Another oral delivery pathway is to dispose the composition in a chewing gum. Particularly where the composition includes an appetite suppressant this is very advantageous in that chewing gum can easily be carried in a pocket or purse and used without advance planning on the part of the subject. In this regard the composition can be made as a liquid encapsulated within an outer layer of chewing gum for faster bio-uptake, or the composition can be entrained somewhat uniformly throughout the matrix of the chewing gum itself.

A further oral delivery pathway is via food, particularly baked goods. Since the compositions described herein aid in smoothing glucose spikes, baking such compositions into a bread or other baked foodstuff is a particularly suitable way to get it into the subject's system at exactly the correct time, without relying on the subject's discipline in curbing their diet or taking a prescription medication at regular intervals. In this regard bread includes conventional loaf-type bread as well as muffins, pitas, tortillas, and the like. Relatedly the compositions described herein can also be made into various pastas.

Another suitable food is prepared desserts, for much the same reasons as above. Many diabetics are unable to control their diet to the degree necessary to lose weight in an amount that will have a noticeable effect on the progression of their disease. By entraining the above QMCa composition with one or more of the above additives in a dessert such as chocolate or ice cream or cakes/cupcakes, the sugar spikes associated with those foods can be effectively managed regardless of the patient's actual diet and even if the patient misses an insulin dose or fails to test his/her own blood sugar level. In this regard chocolate as a dessert includes chocolate bars and candy bars made with chocolate, chips such as may be made into cookies, brownies (in some forms also a cake), chocolate shavings, truffles, frosting on a cake/cupcake and the like which are in a form ready to eat.

In a similar vein the above QMCa composition with one or more of the above additives can be delivered via candy. Particularly advantageous are small candies which typically are individually wrapped in that the patient can eat one or two several times throughout the day to satiate a ‘sweet tooth’ while at the same time the smaller multiple doses of the QMCa composition with additive is more efficiently spread across the hours of the day. Often such candies will be hard such as toffee but some patients may find soft candies such as caramels or nougat or fruit flavored chews more preferable.

Apart from prepared foods such as the breads/pastas, desserts and candies above, the compositions according to these teachings can be delivered via a staple food ingredient, such as granulated or powdered sugar, flour, and cereals such as unprepared oatmeal. In this manner individuals can prepare their own favorite foods in which the compositions described herein would be present through one of the staples they used as an ingredient when they prepared the end food product themselves.

Another pathway for more spaced-in-times dosing is in a beverage such as a carbonated beverage, iced tea or a fruit/sports drink. In this regard the compositions described herein can be pre-mixed in the beverage itself by the bottling company, or it can be in a highly concentrated form with directions to add a few drops to a beverage of the end user's choosing.

Distilled alcohol such as for example whiskey and vodka can cause extreme spikes in blood glucose levels, particularly many popular mixed drinks And so another oral delivery pathway is with alcohol, for example as a mixed drink similar to high-caffeine energy drinks combined with distilled alcohol except in the case of the present compositions the combination with distilled alcohol reduces its adverse glucose effects rather than enhance them. This embodiment includes the compositions combined with distilled spirits in their traditional liquid form as well as compositions in dry form according to these teachings mixed with powdered alcohol. Compositions herein can also be combined with fermented alcohols such as beer or wine for a similar purpose as above, but some experimentation would be necessary so as to obtain a suitable flavor for the combination given that beer and wine generally have less ability to mask additives than distilled spirits.

The individual components of the composition, namely quercetin, myricetin and chlorogenic acid, do not have any direct chemical or physical reaction with the carbohydrates of the food consumed. Rather, the effect on carbohydrate metabolism is due to a direct chemical reaction of the components with the enzymes involved in carbohydrate metabolism. Therefore, the effects are determined by the chemical reaction rate, which in turn is determined by the concentration of the flavonoid components and the components of the enzymatic systems, such as GLUT2 and GLUT4.

In one embodiment, the effective concentration for a composition consisting of a mixture by weight of quercetin, chlorogenic acid and myricetin and quercetin ranges from about 250 mg to about 1000 mg of the composition consumed along with food. In one embodiment, a single dose per day, taken at the beginning of the day, is about 750 mg, or about 1500 mg. In another embodiment, the composition is administered as a dose three times a day in an amount of about 750 mg per dose. The total amount of the composition administered daily, in one embodiment is at least 500 mg, or at least 750 mg, or at least 1000 mg or at least 2500 mg.

When the foodstuff comprises a beverage, the composition may be added to the beverage in an amount of about 0.5 to about 1.5 mg per ml of beverage. When the foodstuff comprises any of the above non-beverage foodstuffs (breads/desserts/staple ingredients/etc.), the composition may be added to these other foodstuffs in an amount of about 2.5 mg to about 10 mg per gram of the non-beverage foodstuff.

While the invention has been explained in relation to various embodiments, it is to be understood that various modifications thereof will be apparent to those skilled in the art upon reading the specification. The features of the various embodiments of the articles described herein may be combined within an article. Therefore, it is to be understood that the invention described herein is intended to cover such modifications as fall within the scope of the appended claims.

What is claimed is:

1. A composition comprising therapeutically effective amounts of quercetin, myricetin and chlorogenic acid in combination with curcumin.

2. The composition according to claim 1, wherein the curcumin is in an amount no greater than 12 g.

3. The composition according to claim 2, wherein the amount of curcumin is greater than 50 mg.

4. The composition according to claim 1, wherein the curcumin is in the form of an extract of turmeric root.

5. The composition according to claim 1, further comprising an agent that increases bioavailability of the curcumin in humans by at least 50%.

6. The composition according to claim 5, wherein the agent includes piperine.

7. The composition according to claim 6, wherein the piperine is in an amount greater than 20 mg.

8. The composition according to claim 1, wherein the agent is in the form of black pepper.

9. The composition according to claim 1, in combination with dried tea leaves.

10. The composition according to claim 9, wherein the composition and the dried tea leaves are combined in a flow through tea bag.

11. The composition according to claim 9, wherein content of the tea bag is characterized by no more than a trace amount of an agent that increases bioavailability of the curcumin in humans.

12. A composition comprising therapeutically effective amounts of quercetin, myricetin and chlorogenic acid in combination with capsicum.

13. The composition according to claim 12, wherein the capsicum is in an amount no greater than 8 g.

14. The composition according to claim 13, wherein the amount of capsicum is greater than 2 g.

15. The composition according to claim 12, wherein the capsicum is in the form of an extract of a hot pepper.

16. The composition according to claim 12, further comprising green tea or an extract thereof.

17. The composition according to claim 16, wherein the green tea or the extract thereof is in an amount greater than 50 mg.

18. The composition according to claim 12, in combination with dried tea leaves.

19. The composition according to claim 18, wherein the composition and the dried tea leaves are combined in a flow through tea bag.

20. The composition according to claim 18, wherein the dried tea leaves are dried green tea leaves.
